(19) United States
(12) Patent Application Publication
(10) Pub. No.: US 2010/0094139 A1
(43) Pub. Date: Apr. 15, 2010

(54) SYSTEM AND METHOD FOR OBTAINING PHYSIOLOGICAL DATA OF A PATIENT

(75) Inventors: Andreas Brauers, Aachen (DE); Xavier Louis Marie Antoine Aubert, Brussels (BE); Ralf Dorscheid, Kerkrade (NL); Frank Johnen, Juelich-Koslar (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/527,884

(22) PCT Filed: Feb. 25, 2008
(86) PCT No.: PCT/IB2008/050664

§ 371 Date: Aug. 20, 2009

(30) Foreign Application Priority Data
Feb. 28, 2007 (EP) 07103237.9

(51) Int. Cl. A61B 5/113; A61G 7/05; A61B 5/11

(52) U.S. Cl. 600/484; 5/600; 600/534; 600/595

(57) ABSTRACT

The present invention relates to a system (1) and method for obtaining cardiopulmonary and/or activity data of a patient (3) situated in a bed (2). In order to provide a simple and reliable technique for obtaining physiological data, in particular cardiopulmonary data and/or activity data, of a patient (3) situated in a bed (2), a method is suggested, comprising the steps of: providing a bed (2) with a bed structure (4) comprising at least one elastically deformable member (6), said member (69 being deformable by the patient (3) being situated in the bed (2); in case of deformation of said member (6), acquiring deformation data (25) of said member (6); and determining physiological data of said patient (3) using said deformation data (25).

The present invention relates to a system and method for obtaining physiological data of a patient. In particular, the present invention relates to a system and method for obtaining cardiopulmonary and/or activity data of a patient situated in a bed.

The measurement of physiological data of a patient, e.g. vital body signs, in a bed using different kind of sensors, e.g. force or pressure sensors, has been known in the prior art for some times. The advantage of these techniques is that the measurement of physiological data is possible without gluing electrodes to the patient's body, or the necessity to wear special sensors, like belts, textiles or the like. However, the integration of those sensors into a bed is either complex or requires specially designed beds. Whereas solutions to overcome this disadvantages has been proposed for hospital beds, for standard beds used at home no solution has been found yet.

It is an object of the present invention to provide a simple and reliable technique for obtaining physiological data, in particular cardiopulmonary data and/or activity data, of a patient situated in a bed.

This object is achieved according to the invention by a method of obtaining physiological data of a patient, comprising the steps of: providing a bed with a bed structure comprising at least one elastically deformable member, said member being deformable by the patient being situated in the bed; in case of deformation of said member, acquiring deformation data of said member; and determining physiological data of said patient using said deformation data.

The object of the present invention is also achieved by a system for obtaining physiological data of a patient, comprising: a bed with a bed structure comprising at least one elastically deformable member, said member being deformable by the patient being situated in the bed; measuring means adapted to acquire deformation data of said member in case of deformation of said member; and data processing and analyzing means adapted to determine physiological data of said patient using said deformation data.

The object of the present invention is also achieved by an elastically deformable member of a bed structure, being connectable to said bed structure, and comprising at least one sensor element for acquiring deformation data of said member, if said member is deformed by the patient being situated in the bed.

The object of the present invention is also achieved by a computer program to be executed in a computer, for obtaining physiological data of a patient; wherein a bed is provided with a bed structure comprising at least one elastically deformable member, said member being deformable by the patient being situated in the bed; and deformation data of said member is acquired in case of deformation of said member; said program comprising computer instructions to determine physiological data of said patient using said deformation data, when the computer program is executed in the computer.

A core idea of the invention is to provide a technique for obtaining cardiopulmonary performance and/or patient activity in a bed using the deformation, in particular the bending, of an elastically deformable member of the bed structure, caused by the patient. In contrast to prior art solutions, where patient movements are measured in a direct way, i.e. by measuring the deformation of a sensor structure, according to the present invention, an elastically deformable member of the bed structure is deformed, and a number of sensors are used to measure the deformation of that member of the bed structure.

According to a main aspect of the invention, form and position of the elastically deformable member, as well as the type of sensor elements are provided in order to achieve a locally resolved force measurement, which is less sensitive to perturbing movements of the patient.

Further aspects of the invention are as follows:
There are numerous force closures between the user's body and the bed frame. Thus, a highly sensitive sensor is required. Quantitative (weight) measurements are not possible nor desired.
The measurement is completely unobtrusive. The sensor is not noticed by the patient.
The measurement can be started and stopped without user interaction. The measurement itself is suited to detect the presence of a person in bed and thus the measurement can be initiated and terminated automatically.
The employed sensors are hidden to the patient and invisible to others, which means an uninvolved person would not see any difference when seeing the bed. This can be important for ill people who do not want to have visible hints to their disease.
The measurement does not pose any complicated requirements to the design of the bed. In a particular embodiment of the invention, the bed merely requires a slatted bed frame.
Additionally, this solution can be realized at low cost. In particular sensor costs and installation costs are low.
The sensor can even be used in double beds as long as separate slatted frames are used, which is common for larger beds.

A bed according to the present invention is defined as a surface or any other device to rest on or to sit on etc., e.g. a conventional bed, a hospital bed, a couch, a conventional chair, a dentist's chair, a wheelchair, an (operating) table, etc. However, the present invention is preferably applicable in home settings. Accordingly the bed is preferably a standard home use bed.

These and other aspects of the invention will be further elaborated on the basis of the following embodiments which are defined in the dependent claims.

With respect to the inventive method the invention comprises the following preferred embodiments: According to a preferred embodiment of the invention the determining step comprises determining of respiratory and/or cardiac activity of the patient. This includes, but is not limited to, extracting of breathing rate and amplitude, pulse rate, and pulse rate variability, from deformation data.

According to another preferred embodiment of the invention the determining step comprises determining of movement activity of the patient. This includes, but is no limited to, determining of patient movements (e.g. turning of the patient), and “presence in bed” data, and calculating of an activity index, from deformation data. Such a determination is preferably be carried out based on analysis of the signal level (presence detection) and amplitude of signal changes (activity index).

According to another preferred embodiment of the invention the determining step comprises evaluating respiratory and/or cardiac performance (cardiopulmonary performance) and/or sleep quality. Such a calculating is preferably carried out by using appropriate signal filtering to separate breathing from cardiac movements and analyze the spectrum of the resulting signals and/or their periodicity in the time domain.

According to yet another preferred embodiment of the invention the method comprises the further step of transmitting results of the determining step to the patient and/or to a remote addressee. For example, the results of the determining step can be communicated to the patient via a display, e.g. via a computer monitor or TV set or the like, or to a remote professional service in order to improve the patient's condition. The term “remote” has to be understood according to the invention as remote with respect to the patient. A remote addressee can be for example a care person in a nearby room or a doctor team in another building or another place of the world.

With respect to the inventive system the invention comprises the following preferred embodiments: According to a preferred embodiment of the invention the elastically deformable member is detachably connected to the bed structure. If the member is detachable, a normal standard bed can easily and quickly converted into a bed for obtaining cardiopulmonary and/or activity data, simply by detaching the member, providing the member with the number of sensors according to the present invention, and assembling the bed structure again. Alternatively, the original member can be replaced by a substitutional member, which already comprises the number of sensor elements. Such a substitutional member can be easily integrated in common slatted bed frame on demand. For replacing the member, no specialized staff is required.

The majority of beds used in private homes are equipped with slatted bed frames or can easily and at low cost be equipped with slatted bed frames. Thus, according to another preferred embodiment of the invention the elastically deformable member is a slat of a slatted bed frame. By using a slat of a slatted bed frame an elastically deformable member is chosen, which enables a locally resolved force measurement, which is less sensitive to perturbing movements of the patient than a corresponding measurement in which another part of the bed would be used as elastically deformable member. In the working position the slat is preferably mounted double-sided and pre-stressed. If instead of a slatted bed frame another type of support is used to carry the mattress, e.g. an elastically deformable grid or web or the like, this other type of support may be used as elastically deformable member according to the invention as well.

In another embodiment of the invention, not the deformation of slats of the slatted bed frame, but the deformation of the frame itself is measured. In other words, the frame serves as elastically deformable member according to the invention. For example, if the frame bears with its four corners on the bed, the weight (and movements) of the patient results in a bending of the frame structure, in particular in a bending of the longitudinal supports of the frame. This can again be measured using sensors, like e.g. strain gauges. In a further embodiment the mattress serves as elastically deformable member according to the invention.

According to another preferred embodiment of the invention the measuring means comprises more than one sensor element. With the use of more than one sensor element the signal to noise-ratio of the measurement is improved. Furthermore, if a larger number of sensor elements are employed, the sensor elements can be arranged in a way that allows a local allocation of deformation data, and therefore a mapping of patient movements. For example, the sensor elements can be located in form of a line or array relative to the surface, on which the patient rests.

According to another preferred embodiment of the invention the sensor elements are attached to and/or integrated into the elastically deformable member. Preferably the sensor elements are glued to the member or connected to the member in another non-detachable way. With such a close connection between sensor and elastically deformable member very precise measurements can be carried out. Additionally, the handling of the member is improved, particular in cases, where the member is a substitutional member.

Preferably, very sensitive force sensors are used in order to detect even small movements of the patient, e.g. cardiac movements. Thus, according to another preferred embodiment of the invention the sensor elements are strain gauges. In this case the sensor elements are preferably positioned in the center of the slats of the slatted bed frame. The resilience of the slat together with the positioning of the sensor elements in the middle of the slat(s) enables a locally resolved force measurement, which is less sensitive to perturbing movements of the patient than a corresponding measurement would be in which the sensor elements would be located in other areas of the bed.

According to another preferred embodiment of the invention the sensor elements are force sensors. In this case the sensor elements are preferably positioned in the mountings of the slats of the slatted bed frame, i.e. near the right and left end of the slat. If the slat is bended by movements of the patient (e.g. by cardiac movements or breathing movements or turning of the patient), the force sensors in the mountings detect the deformation of the slats, in particular in form of a resulting slat movement in the mountings. Such mountings are often made of rubber or the like and are.

Other technical approaches to measuring the deformation of the bed slats are obvious, including optical methods, capacitive measurements, inductive measurements. However, in terms of robustness, low cost and ease of integration the strain gauge setup is preferred.

These and other aspects of the invention will be described in detail hereinafter, by way of example, with reference to the following embodiments and the accompanying drawings; in which:

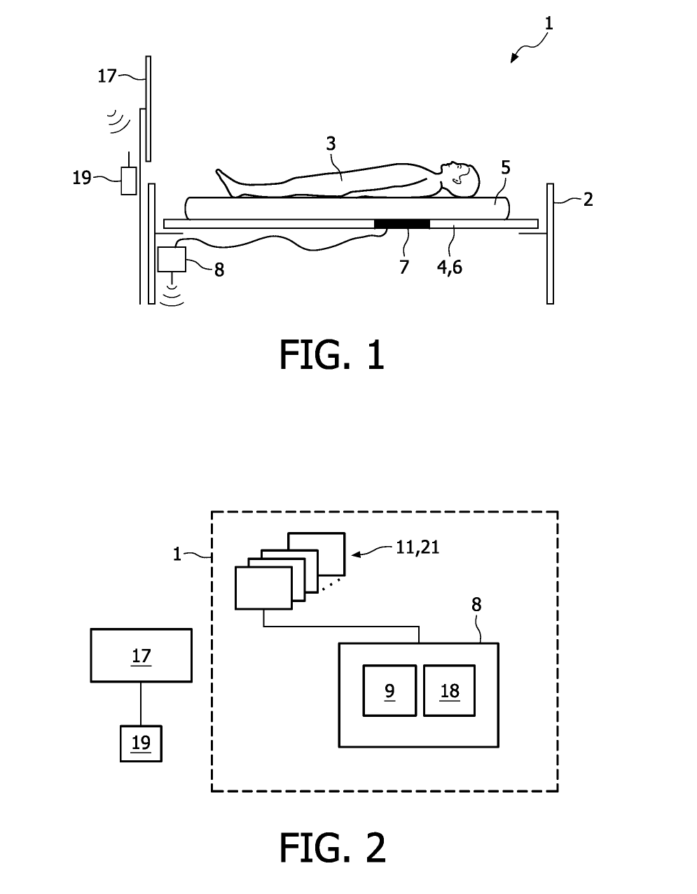

FIG. 1 shows a schematic illustration of a patient situated in a bed according to the invention,

FIG. 2 shows a schematic block diagram of a system according to the invention,

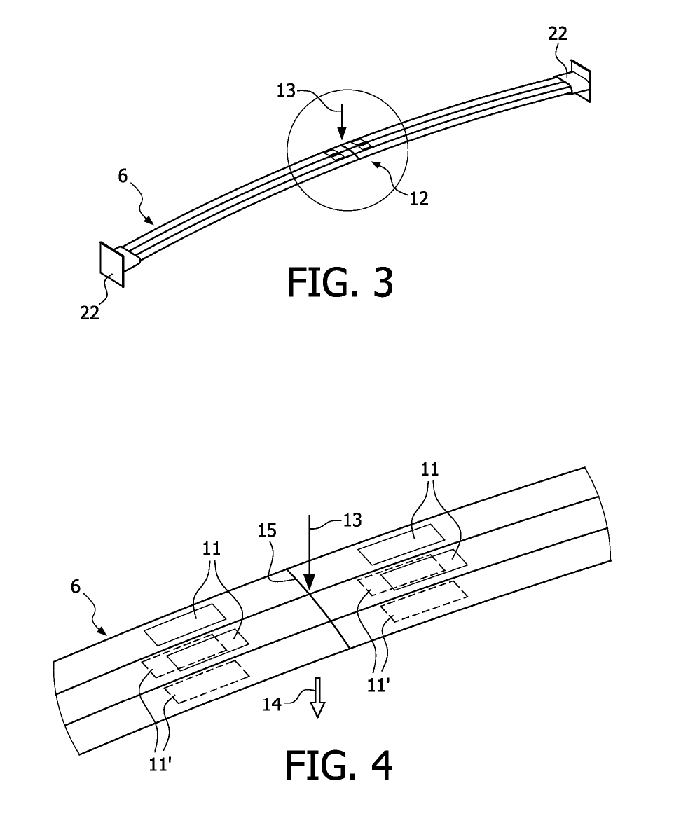

FIG. 3 shows a slat of a slatted bed frame with sensor elements,

FIG. 4 shows a detail of FIG. 3,

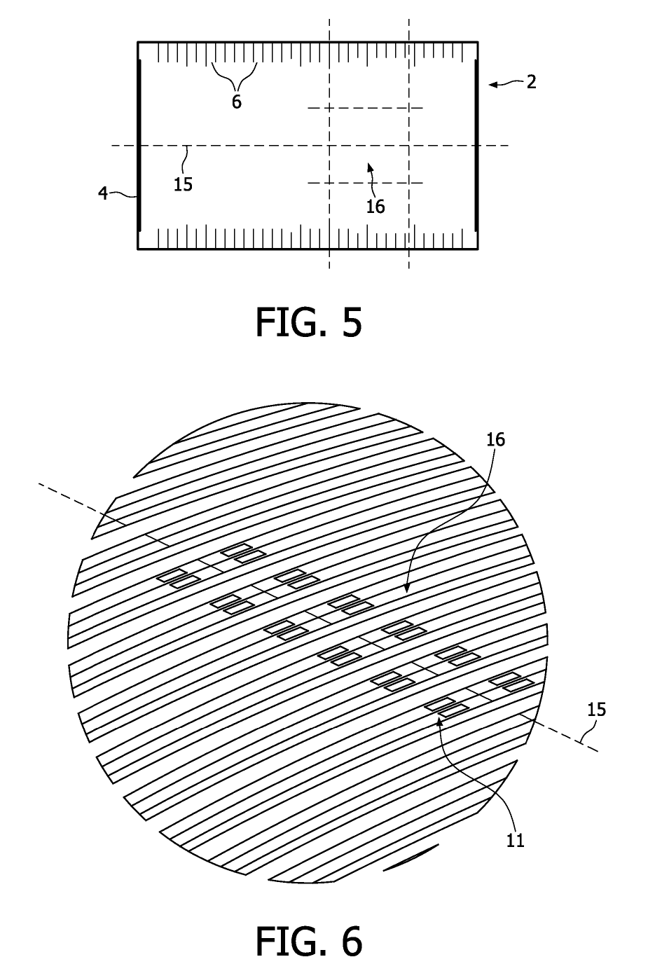

FIG. 5 shows a top view of a bed according to the invention,

FIG. 6 shows a detail of FIG. 5,

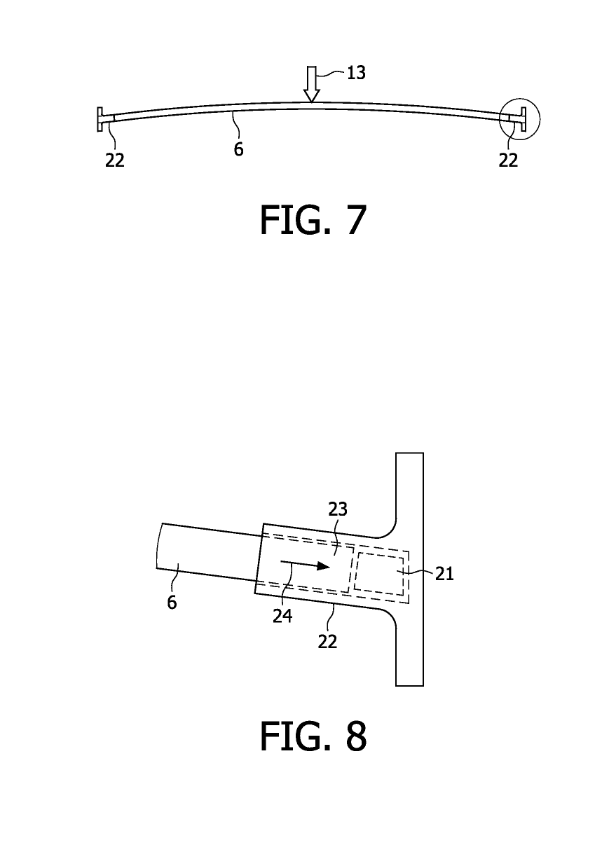

FIG. 7 shows a slat of a slatted bed frame with other sensor elements,

FIG. 8 shows a detail of FIG. 7,

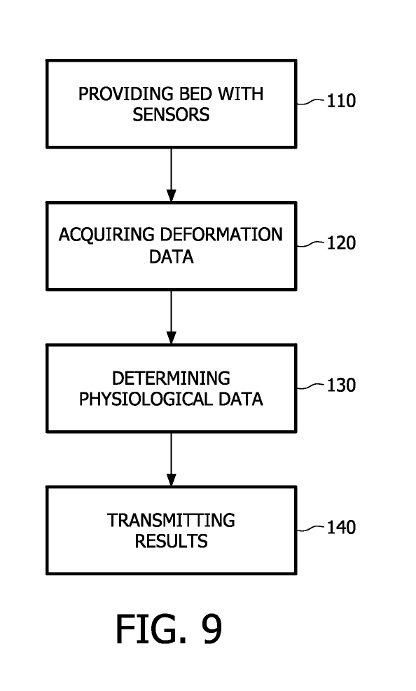

FIG. 9 shows a simplified flowchart of the method according to the invention,

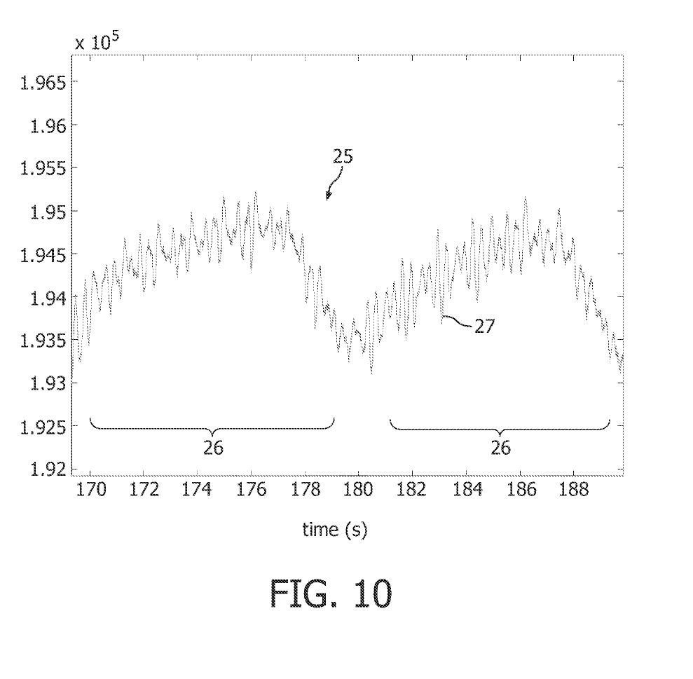

FIG. 10 shows a chart with raw signals from a sensor element, and

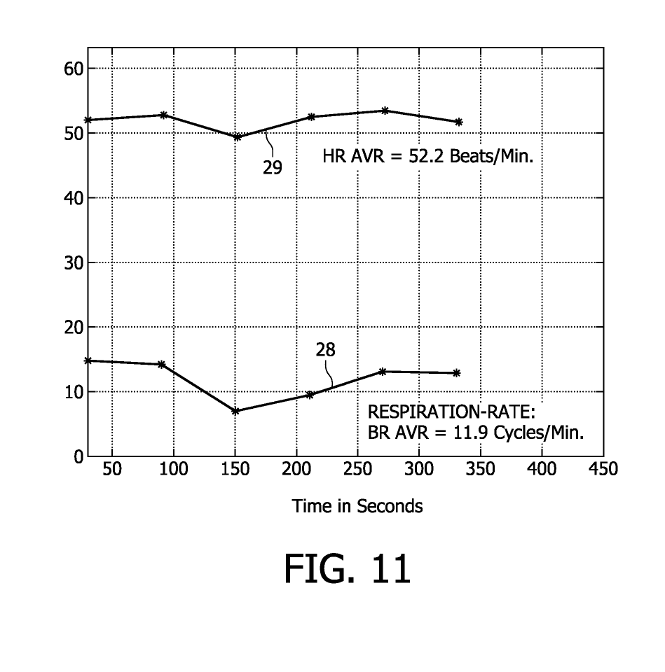

FIG. 11 shows a chart with a heart rate and a breathing rate extracted from a raw signal.

FIG. 1 shows a schematic illustration of a patient 3 situated in a bed 2 according to the invention. The bed 2 is part of a system 1 for obtaining physiological data of a patient 3, as shown in form of a schematic block diagram in FIG. 2. In the illustrated embodiment the bed 2 is a standard bed for home use. The bed 2 comprises a bed structure with a slatted bed frame 4, on top of which a mattress 5 is positioned. The mattress 5 forms the surface, on which the patient 3 is lying. The slats 6 of the slatted bed frame 4 are mounted double-sided and pre-stressed. A number of slats 6 serve as elastically deformable members according to the invention. The slats 6 are deformable by movements of the patient 3, e.g. by cardiac movements or breathing movements of the patient 3 (small movements) or by turning of the patient 3 or the like (large movements). In other words, while the patient 3 is laying on the bed 2, his movements are transferred to mattress 5 and the slatted bed frame 4 and the slats 6 are bending accordingly.

Besides the bed 2, the system 1 further comprises measuring means 7 adapted to acquire deformation data of said slats 6 in case of deformation of said slats 6. The measuring means 7 comprises a number of sensor elements associated to a number of selected slats 6. The measuring means 7 further comprises all necessary wiring in order to connect the sensor elements to each other and/or to connect the sensor elements to other parts of the system 1. The sensor elements are described in more detail below.

The system 1 further comprises a data processing and analyzing module 8 adapted to determine physiological data of said patient 3 using said deformation data. The data processing and analyzing module 8 comprises a processing unit 9, which is adapted for performing all tasks of calculating and computing the measured data as well as determining and assessing results. This is achieved according to the invention by means of a computer software comprising computer instructions adapted for carrying out the steps of the inventive method, when the software is executed in the processing unit 9.

The slats 6, which serve as elastically deformable members, are detachably connected to the slatted bed frame 4. Hence, a normal standard bed can easily and quickly converted simply by replacing a number of original slats by a number of substitutional slats 6, which already comprises sensor elements.

In the embodiment illustrated in FIGS. 3 and 4, strain gauges 11 are used as sensor elements. The strain gauges 11 are positioned in the center 12 of the slats 6 of the slatted bed frame 4. According to the illustrated embodiment, a strain gauge grid 11, 11′ is glued on the surface of a (for example) wooden slat 6, and wired. Preferably, each strain gauge grid is realized as a full bridge with two first strain gauges 11 on the top side of the slat 6 and two other strain gauges 11′ on the opposite (bottom) side of the slat 6. In FIG. 4 the strain gauges 11′ on the bottom side of the slat 6 are shown with dotted lines. This particular setup has the advantage, that the signal to noise ratio is optimized and that the signal is less sensitive to changes in the position of the body. However, other sensor arrangements can be employed as well. In general, a single strain gauge 11 would be sufficient to execute the method according to the present invention.

The strain gauges 11, 11′ are glued to the slats 6 in a way they form integrated slat units, which can easily be handled separately. With such a close connection between strain gauges 11, 11′ and slat 6 very precise measurements can be carried out. Additionally, the handling of the slat 6 is improved, particular in cases of substitution of slats 6. The form and surface of a substitutional slat 6 does not differ from the form and surface of an original slat.

If the slat 6 is bended by movements of the patient 3, e.g. by cardiac movements or breathing movements or turning of the patient 3, in a bending direction 13, the strain gauges 11 detect the deformation of the slats 6, in particular in form of a resulting slat movement in a sensing direction 14, which basically corresponds to the bending direction of the slats 6.

FIG. 5 shows a top view of a bed 2 according to the invention. There large number of slats 6 are indicated schematically. Here, as an example, six slats 6 are equipped with strain gauge grids, so that optimum signals can be extracted regardless of the actual sleeping position of the patient 3, see FIG. 6. In this embodiment 24 top side strain gauges 11 and 24 bottom side strain gauges 11′ are employed in a way that they are arranged symmetrically to the center-line 15 of the bed 2 in an upper region 16 of the slatted bed frame 4, on which the patient's chest normally rests. This is to improve the sensitivity to cardiac and pulmonary movements of the patient. Furthermore, the signal to noise-ratio of the measurement is improved.

According to another preferred embodiment of the invention the sensor elements are force sensors 21. FIG. 8 shows a slat 6 of a slatted bed frame 4 with such a force sensor 21, which in this case is schematically illustrated by dotted lines. The force sensors 21 are positioned inside the rubber mountings 22 of the slats 6 of the slatted bed frame 4. Thus, the force sensors 22 are in working contact with the right and left ends 23 of the slats 6. If the slat 6 is bended by movements of the patient 3, e.g. by cardiac movements or breathing movements or turning of the patient 3, in a bending direction 13, the force sensors 21 in the mountings 22 detect the deformation of the slats 6, in particular in form of a resulting slat movement in the mountings 22 in sensing direction 24. Here, the sensing direction 24 is basically orthogonal to the bending direction 13.

Now, the method of the present invention is explained in more detail. FIG. 9 shows a simplified flowchart of the method according to the invention. First a bed 2 is provided with a bed structure comprising at least one slat 6, as described above (step 110). In case of deformation of said slat 6 by a movement of the patient 3, e.g. by cardiac movements or breathing movements or turning of the patient 3, deformation data of the slat 6 are acquired by means of the measuring means 7, in particular by means of the sensor elements, e.g. the strain gauges 11 and/or the force sensors 21, in a next step 120. Subsequently, physiological data of said patient 3 are determined by means of the data processing and analyzing module 8 and the above described computer program using said deformation data in a next step 130.

In FIG. 10 a chart with a typical raw signal 25 from a strain gauge 11 mounted to a bed slat 6 below the mattress 5 is shown. The raw signal 25 has been measured using a setup as described above. In the chart clearly breathing activity can be seen. Two breathing cycles 26 are shown. Superimposed is a periodic pattern 27 that is attributed to the heart rate (heart pulse).

The determining step 130 comprises processing of raw signals 25 by means of the data processing and analyzing module 8. The processing of the raw signals 25 comprises an appropriate filtering to distinguish between breathing and heart rate. Subsequently the resulting signals are converted by means of the data processing and analyzing module 8 into meaningful information either for the patient 3 or for another person, e.g. a professional like a general practitioner. Preferably the cardiopulmonary activity of the patient 3 is determined in step 130. This includes, but is not limited to, extracting of breathing rate and amplitude, pulse rate, and pulse rate variability, from the raw signals 25.

Additionally or alternatively to the determination of the cardiopulmonary activity of the patient 3, the overall movement activity of the patient 3 is determined. For example, larger patient movements (e.g. turning of the patient) is analyzed and/or an activity index is calculated by means of the data processing and analyzing module 8. The determining step 130 may further comprise evaluating of the patient's cardiopulmonary performance and/or sleep quality.

Finally, the results of the determining step 130 are transmitted to the patient 3 and/or to a remote addressee in step 140. For example, the results of the determining step 130 can be communicated from the data processing and analyzing module 8 to the patient 3 via a display, e.g. via TV set 17, see FIG. 1. For this purpose the data processing and analyzing module 8 comprises a transmitter 18, which is adapted to communicate with a receiver 19 of the TV set 17.

A heart rate and a breathing rate extracted from the raw signals 25, as, for example, transmitted by the data processing and analyzing module 8 to a remote practitioner, is shown in FIG. 11. Here breathing rate 28 and heart rate 29 are shown, which have been calculated from data measured using strain gauges 11 as described above. The strain gauge signal has been recorded in this case during approximately five minutes. During these five minutes the patient 3 lay still. FIG. 11 shows a value for the heart rate 29 every 60 seconds, which is an average of a more frequent heart rate estimation.

All appliances are adapted to carry out the method according to the present invention, as described above. All devices, e.g. the bed 2, the slatted bed frame 4 with the slats 6, the sensors 11, 21 and the data processing and analyzing module 8, are constructed and programmed in a way that the procedures for obtaining data and for data processing run in accordance with the method of the invention.

The processing unit 9 of the data processing and analyzing module 8 may comprise functional modules or units, which are implemented in form of hardware, software or in form of a combination of both. The technical effects necessary according to the invention can thus be realized on the basis of the instructions of the computer program in accordance with the invention. Such a computer program can be stored on a carrier such as a CD-ROM or DVD or it can be available over the internet or another computer network. Prior to executing the computer program is loaded into the computer by reading the computer program from the carrier, for example by means of a CD-ROM or DVD player, or from the internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e.g. using one or more integrated circuits.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 system
2 bed
3 patient
4 slatted bed frame
5 mattress
6 slat
7 measuring means
8 data processing and analyzing module
9 processing unit
10 (free)
11 strain gauge
12 slat center
13 bending direction
14 sensing direction
15 center-line
16 upper region
17 TV set
18 transmitter
19 receiver
20 (free)
21 force sensor
22 mounting
23 slat end
24 sensing direction
25 raw signal
26 breathing cycle
27 heart rate pattern
28 breathing rate
29 heart rate
110-130 method steps

1. A method of obtaining physiological data of a patient (3), comprising the steps of:
providing (110) a bed (2) with a bed structure (4) comprising at least one elastically deformable member (6), said member (6) being deformable by the patient (3) being situated in the bed (2);
in case of deformation of said member (6), acquiring (120) deformation data (25) of said member (6); and
determining (130) physiological data (28, 29) of said patient (3) using said deformation data (25).

2. The method as claimed in claim 1, wherein the determining step (130) comprises determining of respiratory and/or cardiac activity of the patient (3).

3. The method as claimed in claim 1, wherein the determining step (130) comprises determining of movement activity of the patient (3).

4. The method as claimed in claim 1, wherein the determining step (130) comprises evaluating respiratory and/or cardiac performance and/or sleep quality.

5. The method as claimed in claim 1, comprising the further step of transmitting (140) results of the determining step to the patient (3) and/or to a remote addressee.

6. A system (1) for obtaining physiological data of a patient (3), comprising:
a bed (2) with a bed structure (4) comprising at least one elastically deformable member (6), said member (6) being deformable by the patient (3) being situated in the bed (2);
measuring means (7, 11, 21) adapted to acquire deformation data (25) of said member
in case of deformation of said member (6); and
data processing and analyzing means (8, 9) adapted to determine physiological data of said patient (3) using said deformation data (25).

7. The system (1) as claimed in claim 6, wherein the elastically deformable member (6) is detachably connected to the bed structure.

8. The system (1) as claimed in claim 6, wherein the elastically deformable member (6) is a slat (6) of a slatted bed frame (4).

9. The system (1) as claimed in claim 6, wherein the measuring means (7, 11, 21) comprises a number of sensor elements (11, 21).

10. The system (1) as claimed in claim 9, wherein the sensor elements (11, 21) are attached to or integrated into the elastically deformable member (6).

11. The system (1) as claimed in claim 9, wherein the sensor elements are strain gauges (11).

12. The system (1) as claimed in claim 9, wherein the sensor elements are force sensors (21).

13. An elastically deformable member (6) of a bed structure (4), being connectable to said bed structure (4), and comprising at least one sensor element (11, 21) for acquiring deformation data of said member (6), if said member (6) is deformed by a patient (3) being situated in the bed (2).

14. A computer program to be executed in a computer (9), for obtaining physiological data of a patient (3); wherein a bed (2) is provided with a bed structure (4) comprising at least one elastically deformable member (6), said member (6) being deformable by the patient (3) being situated in the bed (2); and deformation data (25) of said member (6) is acquired in case of deformation of said member (6); said program comprising computer instructions to determine (130) physiological data of said patient (3) using said deformation data (25), when the computer program is executed in the computer (9).
